(19) United States
(12) Patent Application Publication
(10) Pub. No.: US 2021/0353903 A1
(43) Pub. Date: Nov. 18, 2021

(54) SOUND SOURCE PROVIDING SYSTEM

(71) Applicant: HA-EUN BIO-HEALTHCARE Inc., Wonju-si (KR)

(72) Inventor: Uen Joo PARK, Seoul (KR)

(21) Appl. No.: 17/064,772

(22) Filed: Oct. 7, 2020

(30) Foreign Application Priority Data
May 15, 2020 (KR) 10-2020-0058443

(51) Int. Cl. A61M 21/02; H04R 1/10; A61B 5/00; A61B 5/053; A61B 5/024

(52) U.S. Cl. CPC A61M 21/02; H04R 1/10; A61M 2021/0027; A61B 5/0533; A61B 5/02405; A61B 5/681

(57) ABSTRACT

The present invention relates to a sound source providing system for stabilizing anxiety symptoms, and more particularly, to a sound source providing system that provides a sound source having a mental and physical stability effect to a person with anxiety symptoms before an exam or anxiety symptoms caused by accidents or stress events.

A sound source providing system according to an embodiment of the present invention comprises: a sensor unit that is worn on a user's body to sense a GSR or HRV; a control unit that receives a GSR or HRV sense signal from the sensor unit and transmits a sound source playback signal; an operation unit for receiving the sound source playback signal from the control unit and playing a sound source such as a tapping sound or a heartbeat sound that is played periodically; and a storage unit that stores the sound source in which the tapping sound or heartbeat sound is recorded.

### CROSS-REFERENCE TO RELATED APPLICATIONS

This application claims priority to Korean Patent Application No. 10-2020-0058443, filed on May 15, 2020 in the Korean intellectual Property Office, the entire disclosure of which is incorporated herein by reference.

### BACKGROUND OF THE INVENTION

### 1. Field of the Invention

The present invention relates to a sound source providing system, and more specifically, to a sound source providing system that can stabilize anxiety symptoms by providing a sound source having a mental and physical stability effect to a person with anxiety symptoms before an exam or anxiety symptoms caused by accidents or stress events.

### 2. Description of the Related Art

Unless otherwise indicated herein, what is described in this section is not prior art to claims of this application, and inclusion in this section is not admitted to be prior art.

Modern people live under various physical and mental stresses, and such stresses are recognized not only as a simple symptom but also as a cause of diseases, and thereby the importance of a method for relieving stresses is emerging. In particular, for those who have anxiety symptoms or depression from specific events caused by trauma or stress, it is of paramount importance to reduce the anxiety symptoms and treat depression. In addition, if a situation similar to the time of an accident occurs, the symptoms become serious due to trauma or mental trauma caused by the accident as they become anxious or suffer severe emotional fluctuations, which can then lead to dangerous accidents.

EMDR exists as a professional and scientific work method to reduce such trauma or stresses. The EMDR is called ‘Eye Movement Desensitization and Reprocessing therapy’, and through horizontal eye movement, symptoms such as depression, panic disorder, post-traumatic stress disorder, dissociation disorder, phobia, and behavioral anxiety are relieved. This therapy, discovered by American psychologist Francine Shapiro, is based on a human psychological phenomenon in which negative and unpleasant thoughts are reduced by spontaneous eye movements.

FIG. 1 illustrates a general state of implementing EMDR therapy. As shown in FIG. 1, EMDR therapy allows a doctor to slowly reciprocate a finger in the horizontal direction left and right, and a patient to follow the doctor's finger with both eyes and watch it, so that a patient naturally moves the eyeball left and right at a constant speed. There is also a content that allows a user to implement EMDR therapy while looking at a computer screen. For example, if a user connects to a specific video-providing site, a user can perform EMDR therapy alone while watching a video. In the EMDR image, a kind of marker object is displayed on a computer screen and continuously moves left and right, and a user observes a marker object on a computer screen to naturally perform eye movement.

In a case of the EMDR therapy, the anxiety symptoms of a patient can be alleviated through eye movements that move the eyes, but it is a method of treating mainly using vision, and among the visual methods, treatment is performed through alternative bilateral light stimulus. However, such a treatment method is mainly based on the above-described treatment method using vision, but a treatment method using hearing or the sense of touch is still insufficient. Among psychological therapy or stress therapy using music is a method of providing therapeutic music that induces a beneficial physiological effect on the human body, a method of treating through music selected according to a user's emotional state, and a method by providing the patient's favorite music. But a therapy method through a bilateral auditory stimulus using hearing which is similar to the aforementioned EMDR therapy of alternative bilateral light stimulus has been relatively less studied than an EMDR therapy.

### PRIOR ART DOCUMENT

### Patent Document

(Patent Document 1) Korean Patent Laid-Open Publication No. 10-2007-0008952 (published on Jan. 18, 2007)

### SUMMARY OF THE INVENTION

It is an object of the present invention to provide a sound source providing system to stabilize anxiety symptoms by delivering sound sources to patients using a bilateral auditory stimulus method.

In addition, it is not limited to the technical problems as described above, and it is obvious that another technical problem may be derived from the following description.

A sound source providing system according to an embodiment of the present invention comprises: a sensor unit that is worn on a user's body to sense a GSR or HRV; a control unit that receives a GSR or HRV sense signal from the sensor unit and transmits a sound source playback signal; a first operation unit for receiving the sound source playback signal from the control unit and playing a sound source such as a tapping sound or a heartbeat sound that is played periodically; and a storage unit that stores the sound source in which the tapping sound or heartbeat sound is recorded.

According to a preferred aspect of the present invention, the first operation unit consists of one selected from earphones worn on a user's ears, headphones, bone conduction earphones, or a speaker installed separately from a user.

According to a preferred aspect of the present invention, the tapping sound or the heartbeat sound is repeatedly played with a predetermined period on the left and right sides of the first operation unit.

According to a preferred aspect of the present invention, the tapping sound or the heartbeat sound is played alternately on the left and right sides of the first operation unit.

According to a preferred aspect of the present invention, the sensor unit is attached to a smart ring worn on a user's finger or a smart band worn on a user's wrist, wherein the smart ring or smart band includes the control unit and the storage unit.

According to a preferred aspect of the present invention, when an abnormal change in a GSR or HRV is sensed by the sensor unit, a sound source is played by transmitting a sound source playback signal from the smart ring or smart band to the first operation unit.

According to a preferred aspect of the present invention, the first operation unit is installed on earrings for fixing glasses formed by wrapping around a user's ears at the end of the glasses worn on a user's face.

According to a preferred aspect of the present invention, the sensor unit is installed on earring parts configured to contact a user's ears in the earrings for fixing glasses.

According to a preferred aspect of the present invention, the tapping sound or heartbeat sound is a sound source for stabilizing anxiety symptoms by being provided to a person with anxiety symptoms due to stress.

According to an embodiment of the present invention, it is possible to help a user feel psychological stability and to heal anxiety symptoms by sensing a GSR or HRV signal from a sensor unit in contact with a body and playing a sound source in a control unit.

Furthermore, according to an embodiment of the present invention, an effect of healing anxiety symptoms can be increased through a simultaneous bilateral auditory stimulus method or a selective bilateral auditory stimulus method.

Furthermore, according to an embodiment of the present invention, an effect of healing anxiety symptoms can be increased through a dual stimulus method of both hearing and the sense of touch by using bone conduction earphones.

The effects of the present invention are not limited to the above effects, and should be understood to include all effects that can be derived from the detailed description of the present invention or the configuration within the scope of the present invention.

### BRIEF DESCRIPTION OF DRAWINGS

FIG. 1 is a conceptual diagram for a conventional EMDR.

FIG. 2 is a conceptual diagram of a sound source providing system according to an embodiment of the present invention.

FIG. 3 is a configuration diagram of a sound source providing system according to an embodiment of the present invention.

FIG. 4 is a diagram of state of use using bone conduction earphones in a sound source providing system according to an embodiment of the present invention.

FIGS. 5a to 5d are graphs illustrating experimental results of an experiment group and a control group in a sound source providing system according to an embodiment of the present invention.

FIG. 6 is a state diagram in which a sensor unit is installed on earrings for fixing glasses in a sound source providing system according to an embodiment of the present invention.

### DETAILED DESCRIPTION OF THE INVENTION

Hereinafter, a configuration, operation and effects of a sound source providing system according to a preferred embodiment of the present invention will be described in detail with reference to the accompanying drawings. For reference, in the drawings below, each component is omitted or schematically illustrated for convenience and clarity, and the size of each component does not reflect the actual size. In addition, the same reference numerals refer to the same elements throughout the specification, and reference numerals for the same elements in individual drawings will be omitted.

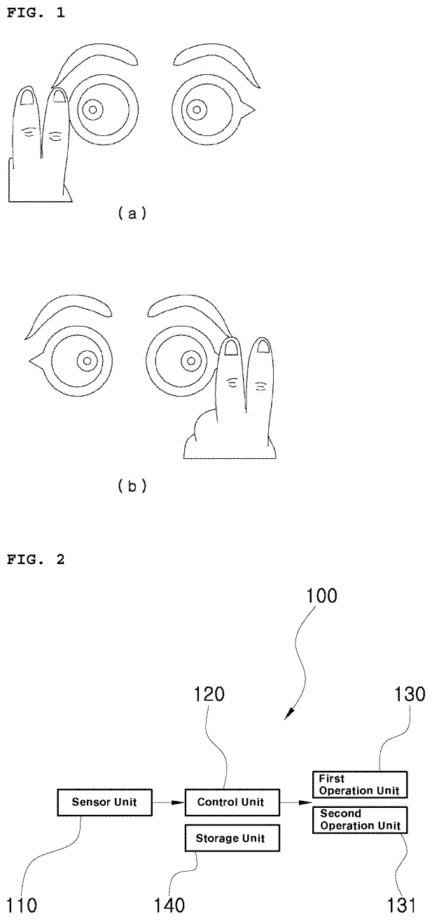

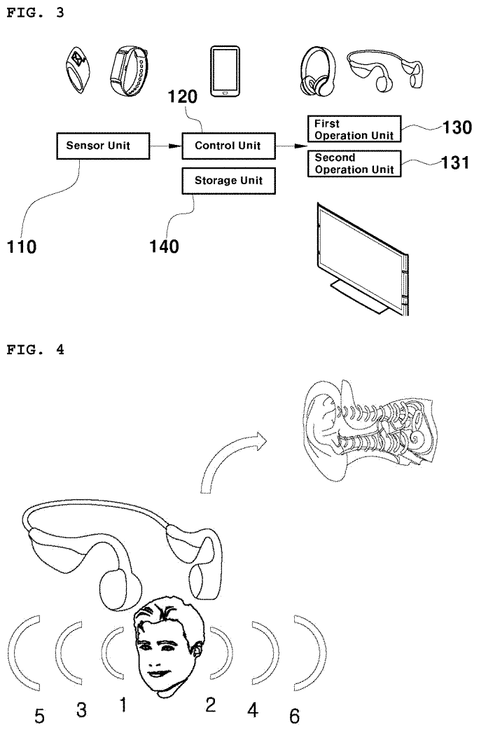

FIG. 2 shows a conceptual diagram of a sound source providing system according to an embodiment of the present invention, FIG. 3 shows a configuration diagram, and FIG. 4 shows a diagram of state of use by using bone conduction earphones.

A sound source providing system 100 according to an embodiment of the present invention comprises: a sensor unit 110 that is worn on a user's body to sense a GSR or HRV; a control unit 120 that receives a GSR or HRV sense signal from the sensor unit 110 and transmits a sound source playback signal; a first operation unit 130 for receiving the sound source playback signal from the control unit 120 and playing a sound source of periodically playing a tapping sound or a heartbeat sound; and a storage unit 140 storing the sound source in which the tapping sound or heartbeat sound is recorded.

The sound source providing system 100 is basically configured to include a sensor attached to a human body, a sound source playing device, and a control unit that controls as a whole, and configurations for each will be described hereinafter.

The sound source providing system 100 includes a sensor unit 110 that recognizes a change in emotion in a human body, and senses a GSR or HRV. Here, the Galvanic Skin Response (GSR) is called an electrical skin reflection, referring that a phenomenon of electrical resistance temporarily decreasing or an action potential occurring by external stimulus or emotional excitement is sensed in the skin of a living body. In addition, the Heart Rate Variability (HRV) is a value capable of confirming a current psychophysiological stable state by checking a human body's autonomic response to stress by analyzing a minute change in heartbeat. The degree of such a phenomenon can be measured by placing an electrode on the skin, amplifying the change in electrical resistance or action potential, and recording it.

Accordingly, when a person's emotion changes due to trauma or stress, a change in a GSR or HRV is sensed at a body part to which the sensor unit 110 is attached.

Next, when the sensor unit 110 senses a GSR or HRV, it transmits the GSR or HRV sense signal to a control unit 120. The control unit 120 may further comprise a storage unit 140 for storing a sound source to be described later, and transmits a sound source playback signal to the first operation unit 130 by receiving a signal from the sensor unit 110, thereby controlling the overall function of allowing the sound source to be transmitted to a person through the first operation unit 130. The control unit 120 may be configured as a portable mobile device including a memory for storing sound sources or the like, and may be configured as a computer such as a desktop or a laptop computer. For example, a user's portable mobile device downloads a sound source through a specific application and stores it in a storage unit 140, and when a signal is transmitted from a sensor unit 110, a sound source playback signal is transmitted to a first operation unit 130, thereby enabling to provide a sound source.

The sound source providing system 100 may further comprise a first operation unit 130 that receives a sound source playback signal from the control unit 120 and plays a sound source in which a tapping sound or heartbeat sound is periodically played. The first operation unit 130 has an output unit such as a speaker through which a sound source can be played, and the output unit can be inserted into a user's ears. Or the output unit has a shape surrounding ears, so that the sound source can be transmitted through the speaker.

In addition, a sound source played by the first operation unit 130 is characterized in that a tapping sound or heartbeat sound is periodically played. The tapping sound has an origin such as a sound that occurs when a person's finger is tapped on the skin during a massage, and may correspond to a sound generated by tapping with a finger to relieve a surprised child.

In addition, the heartbeat is a sound that humans instinctively hear from birth, and there are numerous scientific data on the fact that humans feel psychologically stable through the periodically beating heartbeat. In addition, the storage unit 140 may download and store sound sources for which various therapeutic effects including tapping sound or heartbeat sound are recognized from a device such as a computer, and this may be performed through a portable device application.

When a sound source playback signal is transmitted from the control unit 120, a first operation unit 130 allows the tapping sound or heartbeat sound to be periodically played, preferably at approximately 1 second interval, so that it is possible to help a person who uses the sound source providing system 100 to feel psychologically stable and to heal anxiety symptoms, wherein approximately 1 second interval is associated with a person's heart rate being about 60 times per minute at rest, and the reason why it is not exactly 1 second is that there is a variation of about 20 to 200 msec in the cycle in the heartbeat of a healthy person. More precisely, one tapping sound is given and a tapping sound is used at intervals of a value obtained by adding an arbitrary value between −55 msec to +55 msec per second (here, 55 msec is the average value of the heart rate variability range divided by 2).

According to a preferred aspect of the present invention, the first operation unit 130 consists of one selected from earphones worn on a user's ears, headphones, bone conduction earphones, or a speaker installed separately from a user, wherein the speaker can be used in conjunction with a control unit (mobile multimedia device, mobile phone, PC, laptop, etc.).

In general, as a typical output device for playing a sound source, there are earphones or headphones that are inserted into a user's ears and transmit sound through an eardrum (air conduction method). Recently, bone conduction earphones exist that transmit sound directly to a cochlea (bone conduction method) without going through an eardrum through vibration of bones, Accordingly, the first operation unit 130 is worn on a user's ears and periodically plays the tapping sound or heartbeat sound.

According to a preferred aspect of the present invention, the tapping sound or heartbeat sound is repeatedly played on the left and right sides of the first operation unit 130 with a predetermined period.

The aforementioned EMDR is a method of treating anxiety symptoms through eye movement, and the therapy is performed while alternately giving visual stimulus to the left and right through an alternative bilateral light stimulus, whereas, a sound source providing system 100 according to an embodiment of the present invention is a method of treating anxiety symptoms through a bilateral auditory stimulus using hearing. Therefore, when a sound source playback signal is transmitted from the control unit 120, a tapping sound or heartbeat sound is simultaneously played on the left and right sides of the first operation unit 130 at a predetermined period.

According to a preferred aspect of the present invention, the tapping sound or heartbeat sound is alternately played on the left and right sides of the first operation unit 130.

The aforementioned bilateral auditory stimulus method may be performed by (1) a simultaneous bilateral auditory stimulus method, or by (2) an alternative bilateral auditory stimulus method like an EMDR method. Accordingly, a tapping sound or heartbeat sound played by the first operation unit 130 may be simultaneously played on the left and right sides, or may be played alternately on the left and right sides of the first operation unit 130 as shown in FIG. 4. In this way, the effect of therapy can be increased by selecting an appropriate sound source providing method in accordance with a user's psychological state.

According to a preferred aspect of the present invention, the sound source providing system 100 further comprises a second operation unit 131 for receiving an image playback signal from the control unit 120, and executing a visual stimulus image moving left and right with a predetermined period.

The second operation unit 131 provides a visual stimulus that moves left and right to an arbitrary video viewed by a user according to a user's selection, thereby simultaneously providing visual stabilization stimulus in addition to the auditory stabilization stimulus provided from the first operation unit 130. Here, the second operation unit 131 receiving the image playback signal from the control unit 120 executes the visual stimulus image moving left and right with a predetermined period. For example, when watching a specific learning video or a mental and physical stability meditation video, selecting a visual stabilization stimulus that moves left and right can help to stabilize the mind and body while watching it.

According to a preferred aspect of the present invention, the sensor unit 110 is attached to a smart ring worn on a user's finger or a smart band worn on a user's wrist and the smart ring or smart band includes the control unit 130 and the storage unit 140.

In addition, according to a preferred aspect of the present invention, when a change of more than the stress cutoff in a GSR or HRV is sensed from the sensor unit 110, a sound source is played by transmitting a sound source playback signal from the smart ring or smart band to the first operation unit 130. In this case, a sound source may be set to be automatically played back, after notifying that a user's stress level has been reached, it may be made to determine whether to play or not.

With the recent emergence of various wearable products, wearable products with a function to check the health status by checking a user's heart rate, steps, or breathing status, and to inform them to a user, are also widely used as medical devices such as psychotherapy or depression treatment. Among such wearable products, a smart ring worn on a user's finger or a smart band worn on a user's wrist may be included, and in the present invention, the sensor unit 110 for sensing a user's CSR or HRV is installed on a smart ring or a smart band to sense emotional excitement or mental and physical anxiety through a user's finger or wrist, and transmit the signal to a controller 120, wherein the control unit 120 and the storage unit 140 may be installed in the smart ring or the smart band to play a sound source in a first operation unit 130 linked to communications via Bluetooth or the like. Accordingly, even if there is no configuration such as a separate mobile phone or computer in which the control unit 120 and the storage unit 140 are installed, a GSR or HRV can be sensed and a sound source can be played right away. Accordingly, a first operation unit 130 or a second operation unit 131 may be operated through a configuration such as a mobile phone or a computer in which the control unit 120 and the storage unit 140 are installed, and even without going through this, it is possible to sense a GSR or HRV and play the sound source immediately.

The sound source consists of a tapping sound or heartbeat sound that is played in a simultaneous bilateral auditory stimulus method or in an alternative bilateral auditory stimulus method. In particular, as illustrated in FIG. 4, it has the advantage of increasing the treatment effect of anxiety symptoms by stimulating not only the hearing but also the sense of touch by delivering a sound source through bone conduction earphones.

According to a preferred aspect of the present invention, the first operation unit 130 is installed on earrings for fixing glasses formed by wrapping around a user's ears at the end of the glasses worn on the user's face.

According to a preferred aspect of the present invention, the sensor unit 110 is installed on earring parts configured to contact a user's ears in the earrings for fixing glasses.

Bone conduction earphones and the like have been mentioned as an output device for playing the sound source, but an output device is not limited thereto, and can be applied to glasses worn on a user's face and earrings for fixing glasses that help the glasses be stably mounted on the ears. More specifically, in the case of the bone conduction earphones, a method of transmitting sound directly to a cochlea without passing through an eardrum through the motion of the bone is adopted. The earrings for fixing glasses are in contact with the skin surface around a user's ears. Therefore, when a GSR or HRV signal is sensed as the first operation unit 130 is installed on the earrings, a tapping sound or heartbeat sound from the first operation unit 130 installed on the earrings for fixing glasses through the control unit 120 can be provided to a user. In addition, when the sensor unit 110 is not installed on a smart ring or a smart band, but is installed in an area in contact with the earrings for fixing glasses around the user's ears, a GSR or HRV signal is sensed at the same time as the user feels anxiety symptoms, and a sound source can be played directly from an operation unit 130 of earrings through a control unit 120. By this, it is possible to exhibit an effect of stabilizing anxiety symptoms through sound source playback with only one eyeglass configuration without a separate device in which a sensor unit 110 is installed.

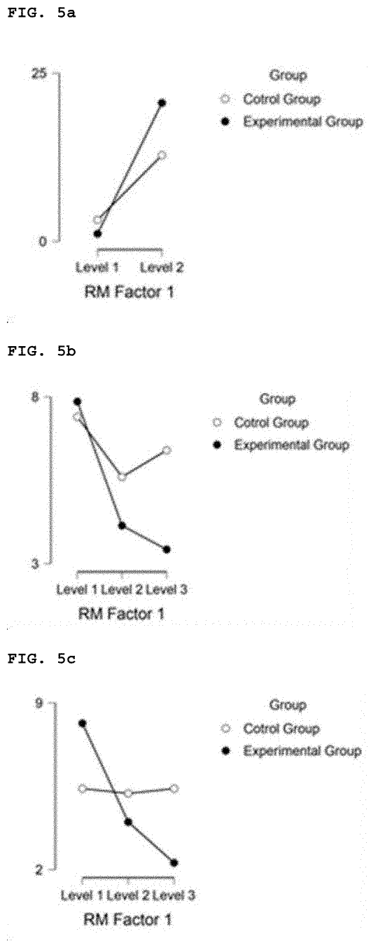

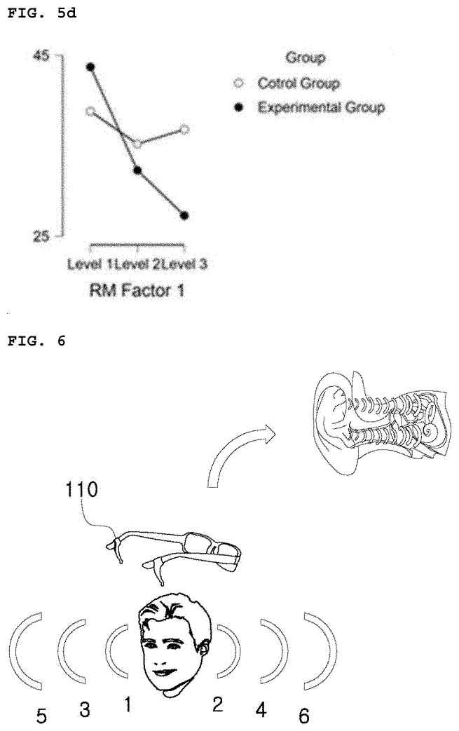

FIG. 5a is a graph illustrating the effect of enhancing learning functions for the sound source providing system according to an embodiment of the present invention through comparative experimental results between an experimental group and a control group.

Hereinafter, the present invention will be described in detail by experimental examples.

However, the following experimental examples are only illustrative of the present invention, and the contents of the present invention are not limited to the following experimental examples.

An experimental group and a control group are divided, and 30 difficult words are solved in the experimental group, and then a sound source is played through a sound source providing system according to an embodiment of the present invention and learning is performed for about 10 minutes. In the control group, the case of learning without providing the sound source providing system was compared.

As a result of performing the second word solving test after word learning through the above process, it can be confirmed that the word correct answer rate of the experimental group was statistically significantly increased. Through this, when a sound source is provided from a sound source providing system according to an embodiment of the present invention, the effect of improving learning ability by reducing psychological anxiety symptoms when performing a task with time pressure is recognized.

Next, FIG. 5b is a graph illustrating the effect of reducing depression for the sound source providing system according to an embodiment of the present invention through comparative experimental results between an experimental group and a control group.

Two groups of adults (experimental group and control group) who recently experienced stress events of SUDs (subjective discomfort) 4 or more were given a first test of PHQ-9-R, a depressive scale, and a sound source was provided through a sound source providing system according to an embodiment of the present invention for 10 minutes, and a second test was performed.

After the second test, the sound source was provided for another 10 minutes, and then the third test was performed. Whenever the sound source was listened to for 10 minutes, a video containing natural scenery and no sound was viewed so that visual attention could be focused. As for the control group, all conditions were the same, but the results obtained by conducting the experiment three times without providing only the sound source are as follows. The experimental group receiving the sound source showed a tendency to rapidly and significantly decrease the feeling of depression associated with the stress event compared to the control group, and it was confirmed that providing the sound source through the sound source providing system according to an embodiment of the present invention has a remarkable effect on reducing depression.

Next, FIG. 5c is a graph illustrating the effect of reducing anxiety for the sound source providing system according to an embodiment of the present invention through comparative experimental results between an experimental group and a control group.

Similar to the effect of reducing depression, two groups of adults (experimental group and control group) who recently experienced stress events of SUDs (subjective discomfort) 4 or more were given a first test of GAD-7-R, an anxiety scale, and a sound source was provided through a sound source providing system according to an embodiment of the present invention for 10 minutes, and a second test was performed. After the second test, the sound source was provided for another 10 minutes, and then the third test was performed. Whenever the sound source was listened to for 10 minutes, a video containing natural scenery and no sound was viewed so that visual attention could be focused. As for the control group, all conditions were the same, but the results obtained by conducting the experiment three times without providing only the sound source are as follows. The experimental group receiving the sound source showed a tendency to rapidly and significantly decrease the feeling of anxiety associated with the stress event compared to the control group, and it was confirmed that providing the sound source through the sound source providing system according to an embodiment of the present invention has a remarkable effect on reducing anxiety.

Furthermore, FIG. 5d is a graph illustrating the effect of stabilizing the level of trauma for the sound source providing system according to an embodiment of the present invention through comparative experimental results between an experimental group and a control group.

An experiment was conducted on the effect of stabilizing the level of trauma through a study similar to the above study on depression or anxiety. Two groups of adults (experimental group and control group) who recently experienced stress events of SUDs (subjective discomfort) 4 or more were given a first test of IES-R, a trauma intensity scale, and a sound source was provided through a sound source providing system according to an embodiment of the present invention for 10 minutes, and a second test was performed. After the second test, the sound source was provided for another 10 minutes, and then the third test was performed. Whenever the sound source was listened to for 10 minutes, a video containing natural scenery and no sound was viewed so that visual attention could be focused. As for the control group, all conditions were the same, but the results obtained by conducting the experiment three times without providing only the sound source are as follows. The experimental group receiving the sound source showed a tendency to rapidly and significantly decrease the degree of shock related to the trauma event compared to the control group, and it was confirmed that providing the sound source through the sound source providing system according to an embodiment of the present invention has a remarkable effect of stabilizing trauma.

Although the present invention has been described with reference to the embodiments shown in the drawings, but these are merely an example. It should be understood by persons having common knowledge in the technical field to which the present invention pertains that various modifications and modifications of the embodiments may be made therefrom. Therefore, it should be understood that the present invention is not limited to the specific embodiments shown and described herein. Accordingly, the real technical protection scope of the present invention is determined by the technical spirit of the appended claims. It also should be understood that the present invention includes all modifications, equivalents, and alternatives falling within the spirit and scope of the present invention as defined by the appended claims.

### DESCRIPTION OF REFERENCE NUMERALS

100: sound source providing system
110: sensor unit
120: control unit
130: first operation unit
131: second operation unit
140: storage unit

What is claimed is:

1. A sound source providing system comprising:
a sensor unit that is worn on a user's body to sense a GSR or HRV;
a control unit that receives a GSR or HRV sense signal from the sensor unit and transmits a sound source playback signal;
a first operation unit for receiving the sound source playback signal from the control unit and playing such a sound source as a tapping sound or heartbeat sound that is played periodically; and
a storage unit that stores the sound source in which the tapping sound or heartbeat sound is recorded.

2. The sound source providing system according to claim 1, wherein the first operation unit consists of one selected from earphones worn on a user's ears, headphones, bone conduction earphones, or a speaker installed separately from a user.

3. The sound source providing system according to claim 1, wherein the tapping sound or heartbeat sound is repeatedly played at a predetermined period on the left and right sides of the first. operation unit.

4. The sound source providing system according to claim 3, wherein the tapping sound or heartbeat sound is played alternately on the left and right sides of the first operation unit.

5. The sound source providing system according to claim 3, wherein the tapping sound or heartbeat sound is simultaneously played on the left and right sides of the first operation unit.

6. The sound source providing system according to claim 1, wherein the sensor unit is attached to a smart ring worn on a user's finger or a smart band worn on a user's wrist, and the smart ring or smart band includes the control unit and the storage unit.

7. The sound source providing system according to claim 6, Wherein a sound source is played by transmitting a sound source playback signal from the smart ring or smart band to the first operation unit when an abnormal change in a GSR or HRV is sensed by the sensor unit.

8. The sound source providing system according to claim 1, Wherein the first operation unit is installed on earrings for fixing glasses formed by wrapping around a user's ears at the end of the glasses worn on a user's face.

9. The sound source providing system according to claim 8, Wherein the sensor unit is installed in earring parts configured to contact a user's ears in the earrings for fixing glasses.

10. The sound source providing system according to claim 1, wherein the tapping sound or heartbeat sound is a sound source for stabilizing anxiety symptoms by being provided to a person with anxiety symptoms due to stress.

11. The sound source providing system according to claim 1, further comprising a second operation unit for receiving an image playback signal from the control unit and executing a visual stimulus image moving left and right with a predetermined period.
